(19) United States
(12) Patent Application Publication
(10) Pub. No.: US 2012/0230936 A1
(43) Pub. Date: Sep. 13, 2012

(54) GEL AIR FRESHENER WITH IMPROVED LENGTH-OF-LIFE AND METHOD FOR PRODUCING SAME

(75) Inventor: Tyler Mikkelsen, Scottsdale, AZ (US)

(73) Assignee: The Dial Corporation, Scottsdale, AZ (US)

(21) Appl. No.: 13/044,152

(22) Filed: Mar. 9, 2011

(51) Int. Cl. A61L 9/04

(52) U.S. Cl. 424/76.4

(57) ABSTRACT

The present invention is a solid and self-standing carrageenan gel air freshener composition that exhibits slowed evaporation and extended length-of-life made possible by the addition of relatively small amounts of extenders consisting of C₁₄-C₁₈ fatty alcohols.

### FIELD OF INVENTION

The present invention relates to an air freshener and in particular to a self-supporting solid gel air freshener comprising extenders for improved length-of-life.

### BACKGROUND OF THE INVENTION

Gel air fresheners have existed in the market and in the patent literature for decades. Such products are generally water-based and may comprise such few ingredients as a gelling/gellation agent (also referred to as a “gellant”), a fragrance oil, and water. The gelling agent may comprise a polymeric material such as naturally marine-sourced carrageenan, agar, or alginate, or it may comprise a complex blend of materials including carrageenan, a natural gum from non-marine sources, a cellulosic material, and various mono- and divalent cations, used together in various combinations to ensure rigidity of the solidified gel and its stability over time against syneresis. An exemplary gel air freshener product based on carrageenan natural gelling agent is Renuzit® Adjustables® Cone Air Freshener marketed by the Dial Corporation, (now Henkel). Preferred gel air fresheners are rigid and “self-supporting.” These products look like solids, although closer inspection reveals they are rigid water gels having an outer surface that is easily penetrated during the procedure used to measure gel strength. Some of the prior art relating to the development of gel-based air fresheners follows.

U.S. Pat. No. 2,691,615 (1954, Turner, et al.) is a very early reference claiming a gel based air freshener. The reference discloses the use of agar-agar, gelatin, pectin, starch, and various gums as potential gelling agents for forming air conditioning gels. The aqueous air treating gel comprised of volatile air treatment compounds, water, and 1 to 4% of an aqueous gelling agent, (preferably agar-agar or calcium alginate), was found to be firm and “substantially devoid of syneresis.”

U.S. Pat. No. 2,927,055 (1960, Lanzet) discloses an air-treating gel comprising water, a volatile air treatment component, and a gelling agent mixture comprising carrageenan, Locust Bean gum, potassium chloride, and sodium carboxymethyl cellulose. The mixture is blended at around 170° F., then poured into molds and cooled. The inventors successfully balanced the amounts of these components to improve the viscosity/handling of the gel in the hot/molten state and to optimize stability, firmness, and appearance of the solidified gel.

U.S. Pat. No. 4,056,612 (1977, Lin) discloses an air freshener gel that utilizes a gelling agent mixture comprising carrageenan (mostly kappa and lambda), Locust Bean gum, and an ammonium salt. The inventive gels exhibited high water gel strengths and syneresis rates of less than 0.3%.

U.S. Pat. No. 4,178,264 (1979, Streit, et al.) discloses an improved air-treating gel composition comprising both carrageenan and a stearate salt used in combination as the gelling agent, wherein the preferred ratio of carrageenan to stearate is from about 0.3:1 to about 5:1. In addition to carrageenan, stearate, water, and volatile actives, a stearate solubility enhancer, such as a solvent or one of a variety of nonionic materials, to increase the solubility of the stearate in the aqueous environment. The preferred components for enhancing the stearate solubility include ethylene glycol, propylene glycol, and ethanol. Most of the Streit example compositions comprise propylene glycol, carrageenan, and sodium stearate combinations for rigid and stable gels.

U.S. Pat. No. 4,666,671 (1987, Purzycki, et al.) discloses fragranced gel blocks useful for deodorizing urinals and toilet bowls. These gel blocks comprise a gelling agent selected from fatty acid salts, sodium alginate, carboxymethyl cellulose, carrageenan, hydroxypropyl cellulose, starches, and gums, although the most preferred gelling agent disclosed is sodium stearate used alone. Solvents including lower alkyl alcohols, diols, and glycol ethers are optionally added to adjust the final melting temperature range of the gel block.

U.S. Pat. No. 5,643,866 (1997, Ansari, et al.) discloses an air-treating gel comprising dibenzylidene sorbitol acetal (DBSA) in combination with a glycol component as the aqueous gelling agent mixture. Such air freshener gels comprising fragrance, water, DBSA and glycol are shaped solid gel products that can withstand temperatures up to 50°-60° C. without melting.

U.S. Pat. No. 5,698,188 (1997, Evans) discloses a gel air fragrancing composition comprising carrageenan in accordance with Lanzet '055. The preferred compositions of Evans comprise 1-20% fragrance, 2-10% carrageenan constituent, and optional preservative and coloring agents, with the balance being water. The carrageenan constituents include commercial thickeners based on carrageenan that are likely to also include proprietary amounts of other materials such as Locust Bean gum, cellulose materials and calcium and/or potassium salts.

Lastly, U.S. Patent Application Publication 2008/0317683 (2008, Trudso) discloses carrageenan compositions and products containing these compositions. The disclosure is directed to methods for extracting and producing carrageenans having a mixture of counter-ions (sodium, potassium, calcium and magnesium), wherein the carrageenan composition has a gelling temperature of between 7° C. and 30° C. An air freshener gel is disclosed that comprises the carrageenan composition having the optimized mixture of cations.

Interestingly enough, in spite of decades of improvements to the physical appearance and the stability of gel air fresheners with concomitant reduction in cost-of-goods, there appears to be no systematic research into ways to improve the longevity (or “length-of-life”) of a gel air freshener. Water-based air freshener gels tend to shrink quickly when exposed to ambient room conditions, with the product no longer emanating perceivable fragrance far before the end of a claimed 30-day length-of-life. Although passive gel air freshener products are relatively inexpensive at retail, there is still consumer disappointment from the rapid evaporation and drying of the product and the rapid decline in fragrance perception. Ideally, retail water-based gel air fresheners having a mass of around 200-300 grams should last at least 30-days and have some physical mass and fragrance perception remaining at 30-days. There remains an unmet need to find simple additives that can be included in an aqueous based gel air freshener composition that will extend the length-of-life of the air freshener, e.g. by slowing overall evaporation, without adversely affecting manufacturability, cost, appearance, fragrance hedonics, or the stability of the product.

### SUMMARY OF THE INVENTION

In a preferred embodiment of the present invention, it has been surprising discovered that the addition of small amounts of long chain fatty alcohol to an aqueous carrageenan-based air freshening gel will slow the evaporation rate and the weight loss of the gel, thus giving the consumer the perception of “increased length-of-life” for the air freshener.

In another preferred embodiment of the present invention, small amounts of C₁₄ to C₁₈ fatty alcohol significantly slows the rate of drying of an air freshener gel exposed to ambient room conditions, giving rise to the perception that the air freshener has an “increased length-of-life.”

### DETAILED DESCRIPTION OF THE INVENTION

The following description is of exemplary embodiments only and is not intended to limit the scope, applicability or configuration of the invention in any way. Rather, the following description provides a convenient illustration for implementing exemplary embodiments of the invention. Various changes may be made in the function, size, and arrangement of the elements described without departing from the scope of the invention as set forth in the appended claims. Changes in shape and size of the overall air freshener do not depart from the intended scope of the invention.

That being said, the compositions of the present invention minimally comprise water, fragrance, a carrageenan gelling agent, and a length-of-life extender. In a preferred embodiment, the air freshener length-of-life extender comprises a long chain fatty alcohol having 10-carbon atoms or more. In the more preferred embodiment, the length-of-life extender comprises a C₁₄-C₁₈ fatty alcohol or blends of fatty alcohols within this chain length range. In order to optimize the physical appearance and stability of the gel air freshener, e.g. to reduce or eliminate syneresis, the air freshener gel in accordance with the present invention may optionally include cellulose materials (e.g. carboxymethyl cellulose, etc.), gums (e.g. Locust Bean, guar, Cassia, xanthan, etc.), synthetic polyacrylate or polyacrylamide polymers, and/or various ionic agents (e.g. various inorganic or organic salts of Na⁺, K⁺, Ca²⁺, Mg²⁺, and the like). Commercially available carrageenan useful in forming air freshener gels may already include a cellulosic substance, a gum, and/or various electrolytes, so that the formulator need not add these substances separately when manufacturing a gel air freshener.

Carrageenans are found in abundance in seaweed. These substances are linear sulfated polysaccharides, and more specifically galactans comprising alternating copolymers of α(1→3)-D-galactose and β(1→4)-3,6-anhydro-D-galacose units differing in the amounts of sulfate ester groups. A number of structurally different carrageenans are known and have been labeled with Greek letters beta, kappa, iota, mu, nu, lambda, theta, and xi for identification. These galactans differ by the percent and location of the ester sulfate groups and by the percent of 3,6-anhydro bonds, structural differences that give rise to differences in their physical properties. Out of these eight natural carrageenans, kappa, iota, and lambda are the most recognizable for industrial use, with ioda and kappa carrageenan the most important for forming rigid gels. Since the natural carrageenans have sulfate groups (i.e. negative charges throughout the polysaccharide molecule), there are cations associated with carrageenan that can include calcium, sodium, magnesium, and/or potassium ions. The relative amounts of these cations may be altered by the supplier during purification, e.g. by using ion exchange processes, or by the formulator during the manufacturing of the gel, (e.g. by adding other salts when the gel mixture is in the heated, molten aqueous state). Kappa carrageenan (25% ester sulfate; 34% 3,6-AG) is the most cation sensitive carrageenan. It is soluble in hot water and forms a strongly rigid and self-supporting gel in the presence of potassium ions, yet forms brittle gels in the presence of calcium ions. Indeed, it is known that the highest gel strength can be obtained when a gel incorporates kappa carrageenan and potassium chloride (KCl) wherein the KCl is used at about 1.6 wt. % and the kappa carrageenan at 1.0 wt. %, (i.e. a 1.6 to 1 weight ratio of KCl to carrageenan). Iota carrageenan (32% ester sulfate; 30% 3,6-AG) forms elastic gels in the presence of calcium ions. Lambda carrageenan (35% ester sulfate; almost no 3,6-AG), on the other hand, is the least cation sensitive carrageenan and is non-gelling. Therefore, for the purpose of forming air freshener gels that are rigid and self-supporting, kappa and iota carrageenan are more preferred. However, and as mentioned above, the precise composition of commercially available carrageenans that find use as gelling agents may not be known with any certainty, and may in fact be of proprietary composition. Various suppliers manipulate the source of the carrageenan by farming specific marine species and they customize the purification processes (alkali treatment, ion exchange, etc.) to produce desired mixtures of carrageenans or pure single carrageenan species such as kappa carrageenan.

With the complexities of natural carrageenan in mind, and being aware of the ability of the suppliers to alter their marine sources and purification methods, the term “carrageenan gelling agent,” an essential ingredient to the extended length-of-life gel air freshener in accordance with the present invention, refers herein to a gelling agent comprised of any carrageenan (i.e. beta, kappa, iota, mu, nu, lambda, theta, and/or xi) in any conceivable proportion. For example, the carrageenan gelling agent for use herein may comprise very pure kappa carrageenan or it may comprise a mixture of only kappa and iota carrageenans. Or the gelling agent of the present invention may comprise a proprietary blend from a carrageenan supplier consisting of one or more carrageenans along with a cellulose material and a salt. Generally speaking, if a commercially available carrageenan is marketed for use as a gelling agent for preparing a solid, rigid gel, it will likely comprise either a majority of kappa-carrageenan or a majority of a mixture of kappa and iota carrageenans, and may include proprietary cellulosic substances, gums, and/or salts. If such additional materials are present in commercial carrageenan marketed for air freshener gels, the materials will likely be in the group of carboxymethyl cellulose, Locust Bean gum, and/or salts such as potassium chloride, sodium chloride, and calcium sulfate.

The carrageenan is used in the present air freshener at an amount sufficient for gellation, which is preferably from about 0.1 wt. % to about 10 wt. %. The carrageenan is most preferably present at about 0.5 wt. % to about 3 wt. %, based on the total weight of the aqueous air freshener gel. As mentioned, the carrageenan for use herein may be any pure carrageenan, or any mixture of any known carrageenans, although preferably carrageenan comprising a majority of kappa carrageenan. Commercial carrageenan that find use herein include, but are not limited to, Grindsted® brand products available from Danisco USA Inc., Danagel® brand products available from FMC, GenuGel® brand products available from CP Kelco, Carrageenan NF from AEP Colloids, Inc., AquaGel® brand products available from Marcel Trading Corporation, and the Eugel® brand products available from MCPI Corporation, particularly Eugel® AFG (“air freshener gel”).

The length-of-life extender preferably comprises a “long chain” fatty alcohol. The relatively subjective term “long chain fatty alcohol” is defined herein to refer to fatty alcohols (linear or branched, saturated or unsaturated) having a total of 10-20 carbon atoms (i.e. inclusive of C₁₀₋₂₀ fatty alcohols, be it saturated or unsaturated, branched or linear). Fatty alcohols for use herein include, but are not limited to, capric alcohol (1-decanol, decyl alcohol, C₁₀H₂₂O), lauryl alcohol (dodecanol, 1-dodecanol, C₁₂H₂₆O), myristyl alcohol (1-tetradecanol, C₁₄H₃₀O), cetyl alcohol (1-hexadecanol, C₁₆H₃₄O), palmitoleyl alcohol (cis-9-hexadecen-1-ol, C₁₆H₃₂O), stearyl alcohol (1-octadecanol, C₈H₃₈O, isostearyl alcohol (16-methylheptadecan-1-ol, branched C₁₈H₃₈O), oleyl alcohol (9-cis-octadecen-1-ol, C₁₈H₃₆O), elaidyl alcohol (9-trans-octadecen-1-ol, C₁₈H₃₆O), linoleyl alcohol (all-cis-9,12-octadecadien-1-ol, C₁₈H₃₄O), linolenyl alcohol (all-cis-9,12,15-octadecatrien-1-ol, C₁₈H₃₂O), arachidyl alcohol (1-eicosanol, C₂₀H₄₂O), gadoleyl alcohol (9-cis-eicosen-1-ol, C₂₀H₄₀O), and 5,8,11,14-eicosatetraen-1-ol (C₂₀H₃₄O). The more preferred length-of-life extenders for use in the present gel air freshener include the following C₁₄-C₁₈ alcohols: myristyl alcohol (1-tetradecanol, C₁₄H₃₀O), cetyl alcohol (1-hexadecanol, C₁₆H₃₄O), palmitoleyl alcohol (cis-9-hexadecen-1-ol, C₁₆H₃₂O), stearyl alcohol (1-octadecanol, C₁₈H₃₈O), isostearyl alcohol (16-methylheptadecan-1-ol, branched C₁₈H₃₈O), oleyl alcohol (9-cis-octadecen-1-ol, C₁₈H₃₆O), elaidyl alcohol (9-trans-octadecen-1-ol, C₁₈H₃₆O), linoleyl alcohol (all-cis-9,12-octadecadien-1-ol, C₁₈H₃₄O), and linolenyl alcohol (all-cis-9,12,15-octadecatrien-1-ol, C₁₈H₃₂O). The most preferred length-of-life extenders for use in the present invention include myristyl alcohol (1-tetradecanol, C₁₄H₃₀O), cetyl alcohol (1-hexadecanol, C₁₆H₃₄O), and stearyl alcohol (1-octadecanol, C₁₈H₃₈O), and mixtures thereof. Cetearyl alcohol is a commercially available mixture of cetyl alcohol and stearyl alcohol (e.g. Lipocol® CS-50 or CS-604 from Lipo Chemicals Inc.) and is also preferred for use herein. The length-of-life extender is preferably used in the gel air freshener composition at from about 0.1 wt. % to about 5 wt. %, and most preferably at from about 0.5 wt. % to about 4 wt. %, based on the total weight of the gel air freshener composition.

The extended length-of-life gel air freshener of the present invention includes a fragrance. A fragrance in accordance with the present invention may comprise one of more volatile organic compounds available from any of the now known, or hereafter established, perfumery suppliers, such as International Flavors and Fragrances (IFF) of New Jersey, Givaudan of New Jersey, Firmenich of New Jersey, etc. Many types of fragrances can be used in the present invention. Preferably the fragrance materials are volatile essential oils. The fragrances, however, may be synthetically derived materials (aldehydes, ketones, esters, etc.), naturally derived oils, or mixtures thereof. Naturally derived fragrance substances include, but are not limited to, musk, civet, ambergis, castoreum and like animal perfumes; abies oil, ajowan oil, almond oil, ambrette seed absolute, angelic root oil, anise oil, basil oil, bay oil, benzoin resinoid, bergamot oil, birch oil, bois de rose oil, broom abs., cajeput oil, cananga oil, capsicum oil, caraway oil, cardamon oil, carrot seed oil, cassia oil, cedar leaf, cedarwood oil, celery seed oil, cinnamon bark oil, citronella oil, clary sage oil, clove oil, cognac oil, coriander oil, cubeb oil, cumin oil, camphor oil, dill oil, estragon oil, eucalyptus oil, fennel sweet oil, galbanum res., garlic oil, geranium oil, ginger oil, grapefruit oil, hop oil, hyacinth abs., jasmin abs., juniper berry oil, labdanum res., lavander oil, laurel leaf oil, lavender oil, lemon oil, lemongrass oil, lime oil, lovage oil, mace oil, mandarin oil, mimosa abs., myrrh abs., mustard oil, narcissus abs., neroli bigarade oil, nutmeg oil, oakmoss abs., olibanum res., onion oil, opoponax res., orange oil, orange flower oil, origanum, orris concrete, pepper oil, peppermint oil, peru balsam, petitgrain oil, pine needle oil, rose abs., rose oil, rosemary oil, sandalwood oil, sage oil, spearmint oil, styrax oil, thyme oil, tolu balsam, tonka beans abs., tuberose abs., turpentine oil, vanilla beans abs., vetiver oil, violet leaf abs., ylang ylang oil and like vegetable oils, etc. Synthetic fragrance materials include but are not limited to pinene, limonene and like hydrocarbons; 3,3,5-trimethylcyclohexanol, linalool, geraniol, nerol, citronellol, menthol, borneol, borneyl methoxy cyclohexanol, benzyl alcohol, anise alcohol, cinnamyl alcohol, (3-phenyl ethyl alcohol, cis-3-hexenol, terpineol and like alcohols; anethole, musk xylol, isoeugenol, methyl eugenol and like phenols; α-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, citral, citronellal, hydroxycitronellal, benzaldehyde, methyl nonyl acetaldehyde, cinnamic aldehyde, dodecanol, α-hyxylcinnamic aldehyde, undecenal, heliotropin, vanillin, ethyl vanillin and like aldehydes; methyl amyl ketone, methyl β-naphthyl ketone, methyl nonyl ketone, musk ketone, diacetyl, acetyl propionyl, acetyl butyryl, carvone, menthone, camphor, acetophenone, p-methyl acetophenone, ionone, methyl ionone and like ketones; amyl butyrolactone, diphenyl oxide, methyl phenyl glycidate, gamma.-nonyl lactone, coumarin, cineole, ethyl methyl phenyl glicydate and like lactones or oxides; methyl formate, isopropyl formate, linalyl formate, ethyl acetate, octyl acetate, methyl acetate, benzyl acetate, cinnamyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, methyl salicylate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, nopyl acetate, β-phenylethyl acetate, trichloromethylphenyl carbinyl acetate, terpinyl acetate, vetiveryl acetate and like esters, and the like. Suitable fragrance mixtures may produce an infinite number of overall fragrance type perceptions including but not limited to, fruity, musk, floral, herbaceous, edible, and woody, or perceptions that are in-between (fruity-floral for example). Typically these fragrance mixtures are compounded by the fragrance houses by mixing a variety of these active fragrance materials along with various solvents to adjust cost, evaporation rates, hedonics and intensity of perception. Well known in the fragrance industry is to dilute essential fragrance oil blends (natural and/or synthetic) with solvents such as ethanol, isopropanol, hydrocarbons, acetone, glycols, glycol ethers, water, and combinations thereof, to make the purchased fragrance raw material blend more easily handled by the formulator and to adjust the rate of evaporation of the volatiles and the hedonics. The preferred fragrance oil for use in the gel air freshener of the present invention may be comprised of a mixture of many fragrance actives and volatile solvents, sometimes along with smaller amounts of emulsifiers, stabilizers, wetting agents and preservatives. More often than not, the compositions of the fragrance oil purchased from the various fragrance supply houses remain proprietary and thus can only be described in general terms.

The fragrance material is preferably incorporated at a level of from about 0.001% to about 5% by weight in the air freshener gel, based on the total weight of the finished composition. Typically the fragrance is added to a rapidly stirred and heated mixture of the carrageenan and water, in which it disperses.

As mentioned above, air freshener gels may also include additional ingredients to increase the stability of the solidified gel. A number of ingredients have been found in the past to synergistically interact with carrageenan to help ensure a solid and stable gel that shows little to no syneresis. Such materials include, but are not limited to: various cellulosic materials like hydroxyethyl cellulose, hydroxypropyl cellulose, and carboxymethyl cellulose, and mixtures thereof; various natural gums derived from non-marine biological sources like gum Arabic, gum ghatti, gum tragacanth, Karaya gum, Guar gum, Locust Bean gum, beta-glucan, Chicle gum, Dammar gum, glucomannan, Mastic gum, Spruce gum, Tara gum, Cassia gum, Gellan gum, and xanthan gum, and mixtures thereof; and various small molecular weight salts like sodium chloride, potassium chloride, magnesium chloride, calcium chloride, sodium acetate, potassium acetate, magnesium acetate, calcium acetate, sodium sulfate, potassium sulfate, magnesium sulfate, and calcium sulfate. The most preferred additives that help produce physically stable carrageenan based air freshener gels include hydroxyethyl cellulose (e.g. Natrosol® from Ashland), carboxymethyl cellulose (AquaCEL® from Ashland), hydroxypropyl cellulose (Klucel® from Ashland), Guar gum (Galactasol® from Ashland), Locust Bean gum (GenuGUM® from CP Kelco), inorganic salts of potassium, calcium, and sodium, and mixtures thereof. Any combination and effective amounts of cellulosic substance, gum, and/or electrolyte may be used as necessary to impart the desired finished properties for the air freshener gel of the present invention. To stabilize a solid gel air freshener of the present invention, the total amount of these optional compounds in the finished air freshener gel may be from about 0.01 wt. % to about 10 wt. %, based on the total weight of the air freshener gel composition.

The gel air freshener in accordance with the present invention may also include dyes, pigments or other suitable colorants to provide aesthetic appeal to the retail gel air freshener product. Such dyes may include FD&C and/or D&C Yellows, Reds, Blues, Greens and Violets, or really any other dye or pigment, and such raw materials are commonly purchased in either powder or liquid form from numerous suppliers. Dyes and/or pigments are incorporated at levels sufficient to provide light color to deep color to the solid gel product. When the optional dyes or pigments are incorporated to produce a colored air freshener gel, they are added at any time into the heated aqueous liquid gel mixture at from about 0.0001% to about 1% by weight, depending on the concentration of the colorants (e.g. if a liquid/diluted dye or a neat powder is the raw material). The water-soluble dyes will dissolve in the water whereas some pigments may only disperse.

The extended length-of-life gel air freshener in accordance with the present invention may also comprise one or more preservatives to help prevent dye fading and/or mold or other microbial growth in and/or on the gel. The preferred microbial preservatives include Neolone® and Kathon® products from Lonza and Rohm & Haas. These materials are incorporated at the manufacturers' recommended levels in the air freshener gel to discourage bacterial and mold growth. An ultraviolet inhibitor and/or an antioxidant such as BHT may also be added to the air freshener gel to reduce dye fading that may become an issue when the air freshener is opened and exposed to light by the consumer.

The extended length-of-life air freshener gel in accordance with the present invention necessarily includes water, and preferably the water is the ingredient present in the largest amount. For example, given the preferred wt. % ranges for the carrageenan gelling agent, the fatty alcohol length-of-life extender, and the fragrance, the remaining wt. % of the composition is mostly water (even with optional adjuvant such as antimicrobials and colorants). Therefore it is preferred that the air freshener gels of the present invention comprise at least 80 wt. % water, and more preferably at least 85 to 90 wt. % water.

### Measurement of the Extension in Length-of-Life for a Gel Air Freshener

“Length-of-life” is a term of art used in the consumer products industry to mean the length of time that an air freshener functions to consumer satisfaction. For air fresheners that comprise a bottle of volatile liquid, the length-of-life equates to the time it takes for the bottle of volatile fragrance to empty. In other words, some air fresheners have an end to their length-of-life, i.e. an “end-of-life” that is readily discernable. Gel air fresheners are passive air fresheners that evaporate at ambient conditions to release volatile fragrances in the surrounding environment such as into a room in a home. Water based gel air fresheners shrink as both the water and the volatile fragrances and other volatile ingredients (if present) evaporate into the air. Invariably some residue remains (e.g. the dried up carrageenan and other non-volatile materials) that obscure any visibly perceivable end-of-life for the air freshener. Therefore it is common in the industry to call that point in time when there is no longer any consumer perceivable fragrance to be the end-of-life for the gel air freshener. Consequently, the length-of-life for a retail gel air freshener then becomes that length of time measured from when the air freshener is first opened and exposed to the ambient air up to the point in time when the consumer can no longer perceive any fragrance. Consumer fragrance perception is usually measured by placing product in a small test room and having consumers come in and smell it. That length of life is preferably about 30-days for a water-based gel air freshener having about 200-300 grams total mass, cast in a container exposed only to ambient conditions (i.e., no mechanical fan blowing on the gel, or any device to heat it above room temperature). Also typical in the industry is to measure ‘weight-loss” of an air freshener. For example, a gel air freshener may have a final weight of dried material that when reached it is known to not emanate any perceivable fragrance for the consumer. In this case, a point on a weight-loss curve (e.g. when the residue weight becomes constant) may be equated to the “end-of-life” of the gel air freshener, and the length-of-life is measured from the start of the weight loss (i.e. when the product is opened and exposed) to the point when the rate of weight loss approaches zero (i.e. the dried residue remains and there is no further possibility of weight loss at ambient conditions). To “extend” length-of-life therefore means herein an extension in the time it takes for a gel air freshener to come to that steady state of dried non-volatile residue (i.e. no additional weight loss). The slowing of the weight-loss has been shown to give a longer perceivable fragrance for the consumer. A slower weight-loss gives the consumer a longer time period of perceivable fragrance from their gel air freshener.

With that being said, the surprising result obtained by the addition of the fatty alcohol length-of-life extender became apparent through weight-loss measurements. As shown in TABLE 1, the rate of weight-loss of a carrageenan air freshener gel was slowed by the addition of 3 wt. % cetyl alcohol. For the experiment, both a control and an extended length-of-life gel air freshener were prepared from the same base composition. The modified air freshener also comprised 3 wt. % cetyl alcohol that was added while the gel composition was still hot and liquid (i.e. before pouring into a mold and cooling). The base composition for the products comprised water, carrageenan (majority kappa), fragrance, a cellulose material and potassium salts. 212 grams of each gel air freshener mixture was cast in a mold and cooled. Each day number indicated, the air fresheners were weighed on an electronic balance. It is evident from the data that the cetyl alcohol slows the weight loss from a water based carrageenan gel air freshener, to the extent that the air freshener will exhibit continued weight loss out to at least 30 days.

TABLE 1
Weight-loss of a gel air freshener with and without cetyl alcohol

Cumulative Weight Lost (in grams)
from a 7.5 oz Air Freshener

| Day No. | Control Gel | Gel with 3 wt. % Cetyl Alcohol |
|---|---|---|
| 1 | 0 | 0 |
| 2 | 11.20 | 7.50 |
| 3 | 22.02 | 15.23 |
| 4 | 31.33 | 22.05 |
| 5 | 40.46 | 28.20 |
| 7 | 66.27 | 48.73 |
| 9 | 84.63 | 63.14 |
| 10 | 94.68 | 71.35 |
| 11 | 101.76 | 77.06 |
| 14 | 125.99 | 97.94 |
| 15 | 133.70 | 105.46 |
| 16 | 138.12 | 110.14 |
| 22 | 168.55 | 144.59 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. (canceled)

2. (canceled)

3. (canceled)

4. (canceled)

5. (canceled)

6. (canceled)

7. (canceled)

8. (canceled)

9. A solid gel air freshener consisting essentially of: (a) from 0.001% to 5% weight percent fragrance; (b) from 0.1% to 10% of primarily kappa carrageenan; (c) from 0.5% to 4% C₁₄-C₁₈ fatty alcohol; (d) from 0.1% to 10% by weight carboxymethyl cellulose; (e) from 0.1% to 10% potassium chloride; and, (f) remainder water.,
wherein the ratio of potassium chloride to kappa carrageenan is about 1.6 to 1.

10. The composition of claim 9, wherein said C₁₄-C₁₈ fatty alcohol consists essentially of cetyl alcohol.
